(19) United States
(12) Patent Application Publication
(10) Pub. No.: US 2011/0027858 A1
(43) Pub. Date: Feb. 3, 2011

(54) Method of Modifying Fungal Morphology

(75) Inventors: Lu-Kwang Ju, Akron, OH (US); Narayanan Srinivasan, Akron, OH (US); Nicholas V. Callow, Girard, OH (US)

Correspondence Address: BROUSE MCDOWELL LPA, 388 SOUTH MAIN STREET, SUITE 500, AKRON, OH, 44311, US

(73) Assignee: University of Akron, Akron, OH (US)

(21) Appl. No.: 12/845,321

(22) Filed: Jul. 28, 2010

Related U.S. Application Data
(60) Provisional application No. 61/229,045, filed on Jul. 28, 2009.

(51) Int. Cl. C12N 9/42; C12N 1/14

(52) U.S. Cl. 435/209; 435/254.1; 435/256.1; 435/256.3; 435/256.7; 435/256.8

(57) ABSTRACT

Provided is a method of modifying fungal morphology. The method comprises introducing an amount of a surfactant to a fungus, or to a growing mixture comprising a fungus, wherein said amount of a surfactant is sufficient to induce a change in the morphology of said fungus.

This application claims priority to U.S. Ser. No. 61/229,045 titled METHOD OF MODIFYING FUNGAL MORPHOLOGY, filed Jul. 28, 2009, which is incorporated herein by reference.

### TECHNICAL FIELD

Provided is a method of modifying fungal morphology and/or enzyme production. More particularly, provided is a method of modifying morphology and/or enzyme production of microscopic fungus, by introduction of a surfactant in order to improve the efficiency of production of fungal products.

### BACKGROUND

Microscopic growing in the form of filaments may comprise, without limitation, molds. Fungal products may comprise any product produced by a fungus. Fungal products may comprise cellulase. Cellulase comprises a group of enzymes that may catalyze the hydrolysis of cellulose. Without limitation, cellulose may comprise polysaccharides and bagasse. Hydrolysis of cellulose may produce monomeric sugar units, such as glucose. Cellulase may comprise a group of enzymes that act synergistically to break down the sugar polymers in the plant biomass into monomeric sugar units. Cellulase may comprise endo-glucanase, cellobiohydrolase, β-glucosidase. Endo-glucanase may comprise EG, carboxymethyl cellulase, and Cx. Cellobiohydrolase may comprise CBH, exo-glucanase, and C1. β-Glucosidase may comprise cellobiase.

Cellulose may be used as a raw material for producing many desired intermediate and/or end products. The glucose subunits of cellulose can be used, without limitation, to produce many desired intermediate and/or end products. Cellulose processing technology has encountered difficulty in the efficient and/or controlled break-down or modification of cellulose and/or cellulose-containing materials and products. Cellulase-catalyzed hydrolysis of cellulose can promote the break-down or modification of cellulose. Accordingly, economical production of cellulase is a matter of interest.

A number of fungal species are known to produce cellulase, including, without limitation, Trichoderma reesei RUT C30, other strains of Trichoderma reesei, Trichoderma viride, Trichoderma koningii, Acremonium cellulolyticus, Aspergillus acculeatus, Aspergillus fumigatus, Aspergillus niger, Irpex lacteus, Penicillium funmiculosum, Phanerochaete chrysosporium, Schizophyllum commune, Sclerotium rolfsii, Sporotrichum cellulophilum, Talaromyces emersonii, and Thielavia terrestris. Some mycelial actinomycetes such as Streptomyces sp., Thermoactinomyces sp., and Thermomonospora curvata also produce cellulase. Unless otherwise noted, as used herein, the terms fungus, fungi and fungal will comprise, without limitation, the above-noted fungi, any other fungi, mycelial actinomycetes, and filamentous actinomycetes. Hereafter, unless otherwise noted, Trichoderma reesei RUT C30 will be referred to as T. reesei or Trichoderma.

T. reesei can produce cellulase extracellularly. That is, the cellulase produced by T. reesei may be found as soluble or dissolved material in the environment in which the fungus is grown. The cellulase enzymes produced by T. reesei may include a mixture of endo-glucanase, exo-glucanase, and β-glucosidase.

T. reesei is a fungus having a naturally filamentous morphology in submerged cultivation. Unless otherwise modified, a fungus having a filamentous morphology, such as, without limitation, T. reesei, grows as filaments. Fungal filaments are elongated strands. Fungal filaments may be elongated and branched strands. A growth medium and a fungus cultured therein may be referred to collectively as a growing mixture. As it grows, a fungus having a filamentous morphology tends to increase the viscosity of the growing mixture. A fungus having a filamentous morphology tends to settle only slowly when the growing mixture is not mixed and the settled fungal cells tend to pack very loosely; the settled cellular mass tends to have a low cellular density. High viscosity growing mixtures, slow settling, and loose cell packing each can complicate collection of product-bearing cell-free medium.

Higher viscosities in growing mixtures may present some issues for the production of fungal products. These issues may comprise oxygenation issues and harvesting issues.

In the growth of fungi that consume oxygen from the growing mixture for continued growth, it is possible to introduce oxygen into the growing mixture by bubbling a gas comprising oxygen, such as, without limitation, air or oxygen, therethrough. The oxygenation rate may be further increased by mechanically mixing or agitating the growing mixture. T. reesei is a fungus that consumes oxygen from the growing mixture for continued growth. If the oxygen consumed by a fungus of a growing mixture is not replaced at a rate equal to or greater than the rate of consumption, the oxygen needed to sustain the existing fungus and to sustain growth of additional fungus will begin being depleted. In lower viscosity growing mixtures, the rate of oxygenation by agitation and bubbling a gas comprising oxygen therethrough can be quite high and oxygen can be readily supplied to sustain the existing fungus and to support growth of additional fungus. As the viscosity of the growing mixture increases, oxygenation issues may appear. As the viscosity of the growing mixture increases, the rate of oxygenation from agitation and bubbling a gas comprising oxygen therethrough may diminish. In some embodiments, diminishment of the rate of oxygenation due to increased viscosity results in oxygen input rates equal to or lower than oxygen consumption rates by the fungus, such that additional growth will be unsustainable. In some embodiments, diminishment of the rate of oxygenation due to increased viscosity results in oxygen input rates too low to sustain the existing fungus and some of the fungus will begin to die. In some embodiments, the diminishment of the rate of oxygenation due to increased viscosity presents an undesirable limit to the growth of the fungus. Higher viscosity growing mixtures may introduce limits to growth that reduce efficiency. Methods to reduce viscosity or prevent viscosity increases in fungal growing mixtures may promote efficiency in production of fungal products, such as, without limitation, cellulase.

In the growth of fungi that produce a fungal product soluble in the growth medium, harvesting of the fungal product may comprise separation of the growing mixture into the constituent fungus and growth medium. As noted above, T. reesei is a fungus that produces a fungal product that is soluble in the growth medium. At low viscosities, separation of the growing mixture into the constituent fungus and growth medium may comprise decanting, screening, filtration, settling, other simple separation processes, or centrifugation. As viscosity increases in the growing mixture, harvesting issues appear. As the viscosity of the growing mixture increases, it becomes more difficult to separate the growing mixture into the constituent fungus and growth medium. At higher viscosities, separation becomes more complex, taking longer or requiring more complex equipment, or both. At some viscosities, the separation processes needed to separate the mixture in a timely manner may become complex or expensive. At some viscosities, vacuum filtration or centrifugation is necessary to separate the mixture in a timely manner. Higher viscosity growing mixtures introduce harvesting complexities that reduce efficiency. Methods to reduce viscosity or prevent viscosity increases in fungal growing mixtures may promote efficiency in production of fungal products, such as, without limitation, cellulase.

As noted above, the filamentous morphology of fungal cells may also produce fungal cells tend to pack very loosely into cellular collections that have a low cellular density. Without wishing to be bound to any particular theory, it is surmised that a branched filament structure diminishes interpenetration of the filaments and diminishes reduction of the porosity in the structure. Fungal cells that tend to pack very loosely may present some issues for the production of fungal products. These issues may comprise harvesting issues.

It remains desirable to develop efficient methods to produce cellulase. Methods to promote efficiency in production of fungal products, such as, without limitation, cellulase, may include methods to modify the morphology of the fungus.

### SUMMARY

Provided is a method of modifying fungal morphology. The method comprises introducing an amount of a surfactant to a fungus, or to a growing mixture comprising a fungus, wherein said amount of a surfactant is sufficient to induce a change in the morphology of said fungus.

Also provided is a method of producing cellulase. The method comprises providing a growth medium, fungal cells, and a surfactant and mixing the fungal cells, the growth medium, and an amount of the surfactant to form a mixture. The amount of the surfactant is an amount sufficient to induce a change in the morphology of the fungal cells.

Also provided is a growing mixture. The growing mixture comprises a fungus, a growth medium, and an amount of a surfactant sufficient to induce a change in the morphology of the fungus.

### BRIEF DESCRIPTION OF THE DRAWINGS

The present subject matter may take form in certain parts and arrangement of parts, embodiments of which will be described in detail in this specification and illustrated in the accompanying drawings which form a part hereof, and wherein:

FIG. 1 is a flow-chart illustrating one embodiment of a process for growing a fungus of modified morphology;

FIG. 2 is a flow-chart illustrating one embodiment of a process for harvesting growth medium and retaining grown fungus for reuse;

FIG. 3 is a microscopic picture of a fungus having naturally filamentous morphology comprising filaments;

FIG. 4 is a microscopic picture of a fungus having naturally filamentous morphology comprising filaments forming pellet structures and forming bulges at the tips thereof;

FIG. 5 is a picture of vials containing growing mixtures comprising various surfactant concentrations;

FIG. 6 is one embodiment of a Petri dish containing a growing mixture;

FIG. 7 is another embodiment of a Petri dish containing a growing mixture;

FIG. 8 is another embodiment of a Petri dish containing a growing mixture;

FIG. 9 is another embodiment of a Petri dish containing a growing mixture;

FIG. 10 is a graph showing mean pellet diameter and standard deviation of pellet diameter for multiple embodiments of growing mixtures;

FIG. 11 is a graph showing cellulase, sugar, rhamnolipid, and intracellular protein data as a function of time in a growing mixture;

FIG. 12 is a graph showing cellulase production as a function of time in a series of experiments;

FIG. 13 is a diagram of a rhamnolipid molecule;

FIG. 14 is another diagram of a rhamnolipid molecule;

FIG. 15a shows one embodiment of a container of a growing mixture of T. reesei Rut C-30 with 1.0 g/L rhamnolipid;

FIG. 15b shows one embodiment of a container of a growing mixture of T. reesei Rut C-30 with 1.0 g/L Triton X-100;

FIG. 15c shows one embodiment of a container of a growing mixture of T. reesei Rut C-30 with no surfactant added;

FIG. 16 shows a graph describing various parameters in a growth over time in one embodiment;

FIG. 17 shows a graph describing certain growth parameters in a certain embodiment;

FIG. 18 shows a graph describing certain growth parameters in a certain embodiment;

FIG. 19 shows a graph describing certain growth parameters in a certain embodiment;

FIG. 20 shows a graph describing certain growth parameters in a certain embodiment;

FIG. 21 shows a graph describing certain growth parameters in a certain embodiment; and

FIG. 22 shows a graph describing certain growth parameters in a certain embodiment.

### DETAILED DESCRIPTION

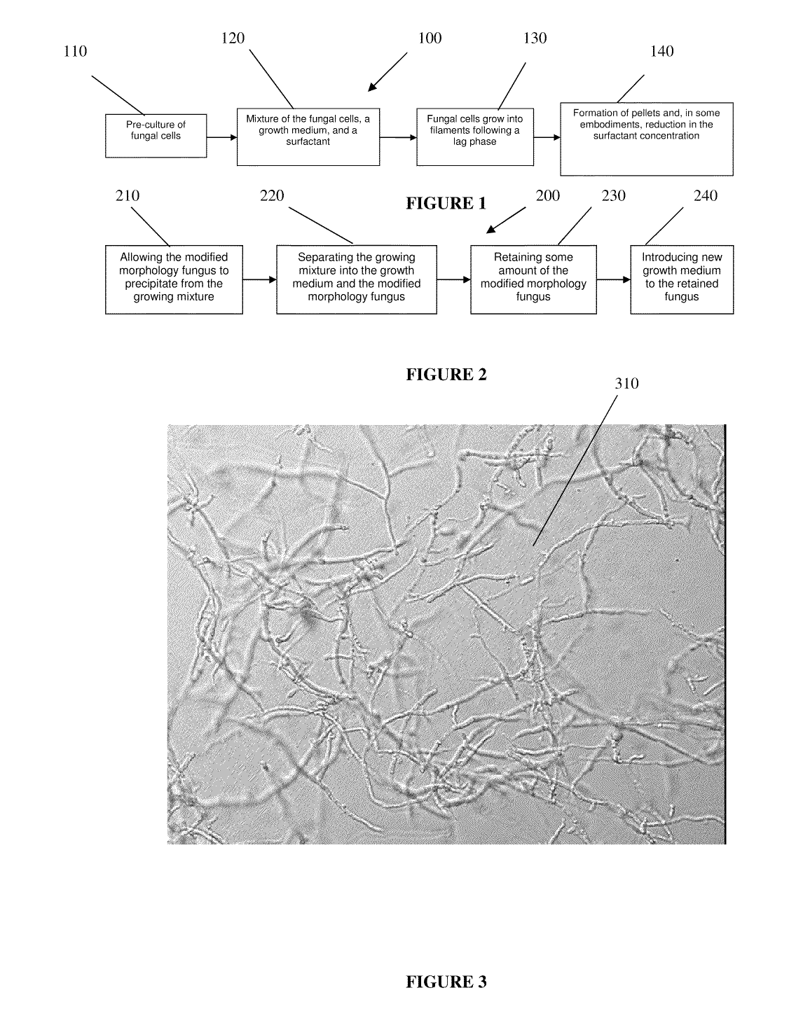

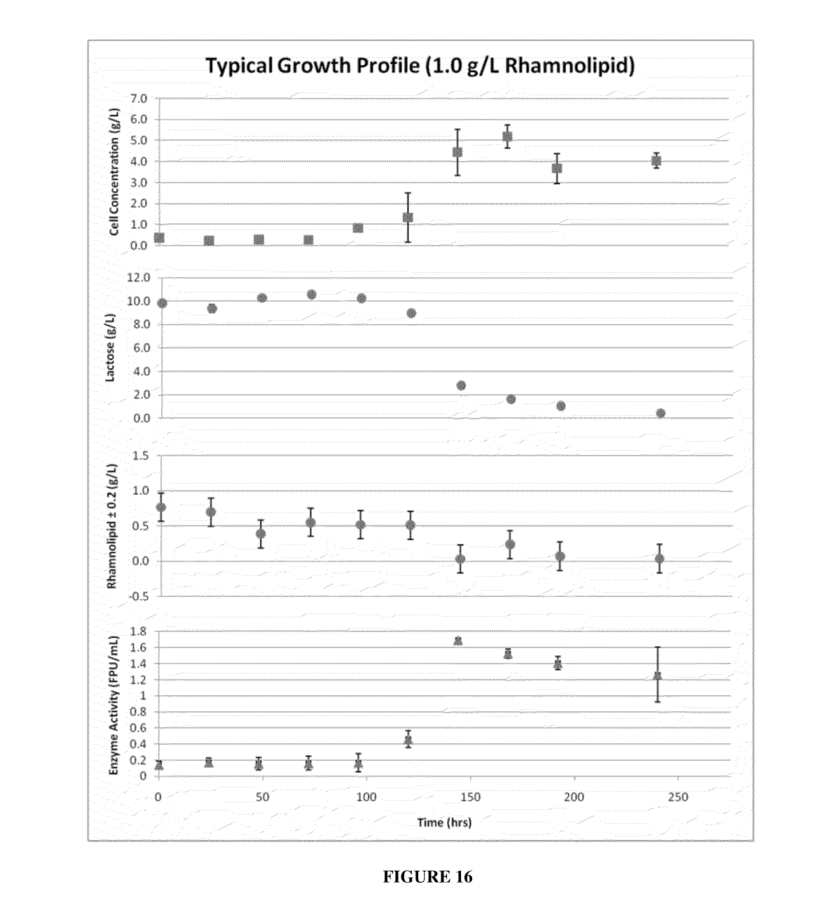

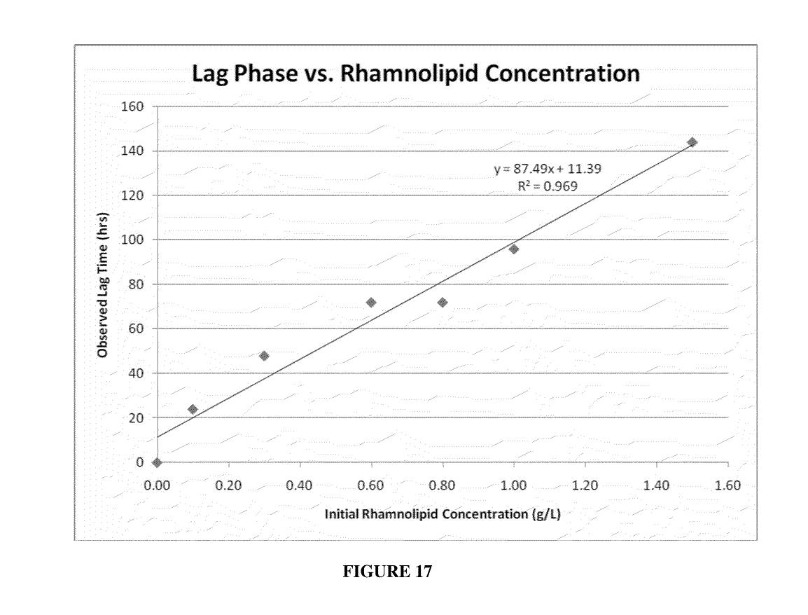

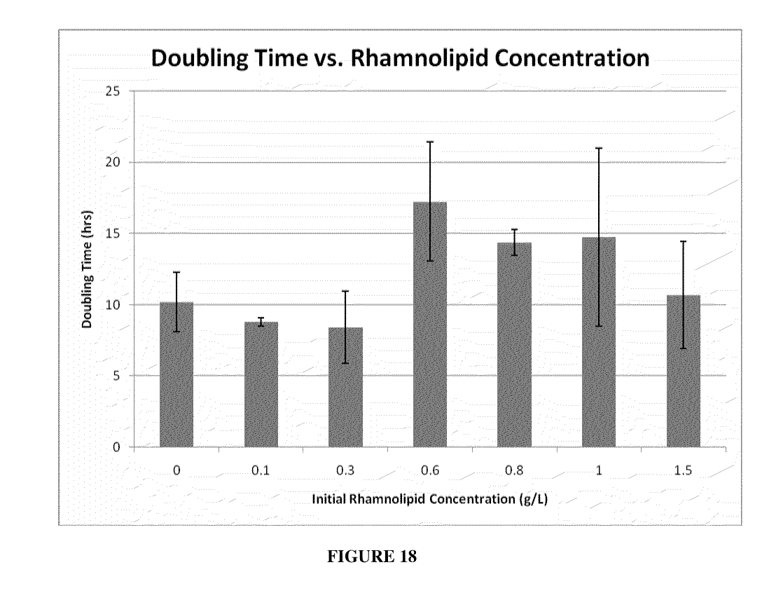

Reference will be made to the drawing, FIGS. 1-22, wherein the showings are only for purposes of illustrating certain embodiments of a method of modifying fungal morphology, and not for purposes of limiting the same. Specific characteristics relating to the embodiments disclosed herein are not to be considered as limiting, unless the claims expressly state otherwise.

In certain embodiments, cellulase is produced by growing a fungus having a naturally filamentous morphology and being capable of producing cellulase. Fungi capable of producing cellulase having a naturally filamentous morphology may comprise, but are not limited to, T. reesei, Trichoderma viride, Trichoderma koningii, Acremonium cellulolyticus, Aspergillus acculeatus, Aspergillus fumigatus, Aspergillus niger, Irpex lacteus, Penicillium funmiculosum, Phanerochaete chrysosporium, Schizophyllum commune, Sclerotium rolfsii, Sporotrichum cellulophilum, Talaromyces emersonii, and Thielavia terrestris. FIG. 3 shows a highly magnified view of the filaments 310 of a fungus having a naturally filamentous morphology.

In certain embodiments, growth of a fungus with naturally filamentous morphology in a growth medium results in increasing viscosity of the growing mixture. In certain embodiments, growth of a fungus with naturally filamentous morphology in a growth medium will increase the viscosity to the point that, by the time fungal products are ready to harvest, the growing mixture has a high viscosity. Without limitation, in some embodiments, production of fungal products in a growing mixture comprising a fungus with naturally filamentous morphology and having a low viscosity at harvest may be achieved by modifying the morphology of the fungus.

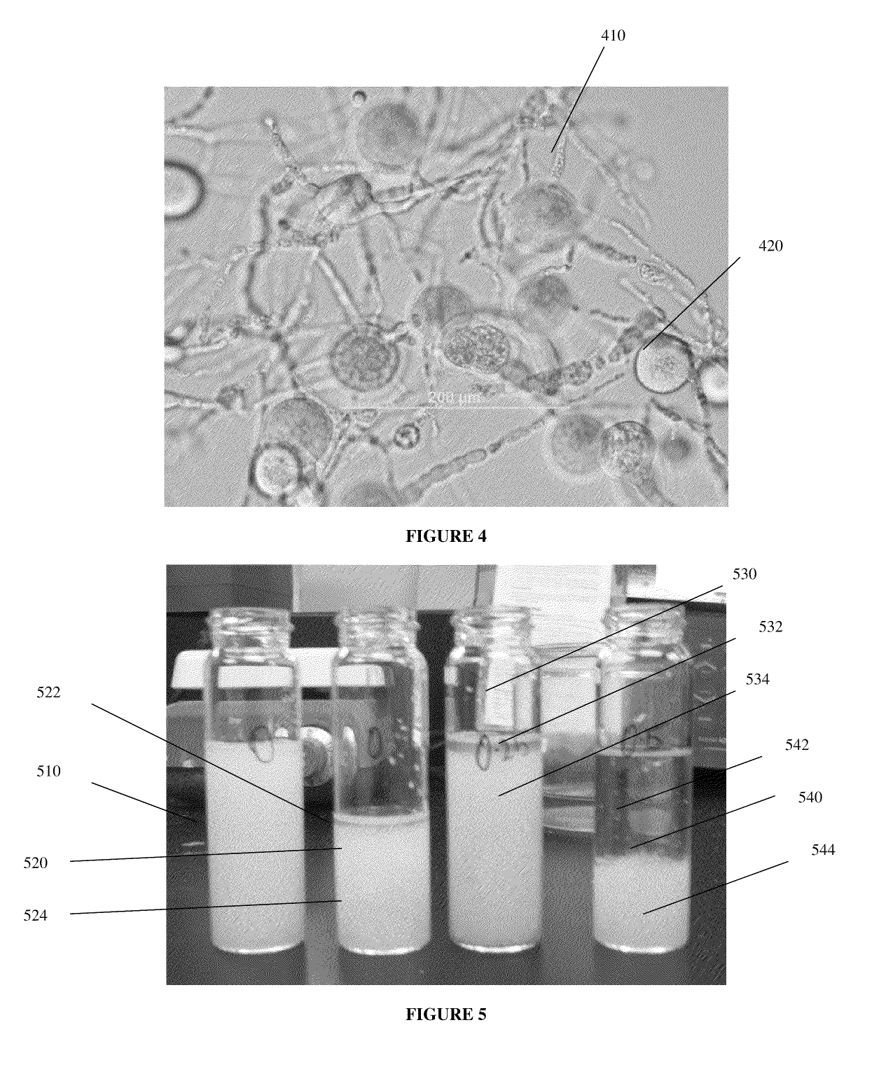

In some embodiments the morphology of a fungus may be modified by mixing a surfactant with the fungus or with a growing mixture comprising the fungus. Without limitation, the morphology of T. reesei may be modified by introducing a surfactant to a growth medium comprising the T. reesei. FIG. 1 shows a method 100 for modifying the morphology of a fungus with naturally filamentous morphology by mixing together fungal cells, a growth medium, and a surfactant. Method 100 comprises pre-culturing the cells of the fungus with naturally filamentous morphology 110. Method 100 comprises mixing together the pre-cultured cells of the fungus with naturally filamentous morphology, a growth medium, and a surfactant 120. The resulting mixture will have some surfactant concentration. Method 100 comprises allowing the pre-cultured cells of the fungus with naturally filamentous morphology to grow into filaments following a lag phase 130. Method 100 comprises allowing the cells of the fungus with naturally filamentous morphology to form pellets 140. Without limitation, in some embodiments, the formation of pellets 140 in method 100 comprises the formation of microscopic bulges on the filaments. Without limitation, in some embodiments, the formation of pellets 140 in method 100 does not comprise the formation of microscopic bulges on the filaments. Without limitation, in some embodiments, the formation of pellets 140 in method 100 occurs contemporaneously with a reduction in the biosurfactant concentration in the growth medium. FIG. 4 shows a highly magnified view of the filaments of a fungus 410 having a naturally filamentous morphology wherein the morphology of the fungus has been modified and wherein that at least some of filaments 410 comprise bulges 420.

Without limitation, a surfactant may comprise a biosurfactant or a synthetic surfactant. Without limitation, a biosurfactant may comprise, a rhamnolipid. Without limitation a synthetic surfactant may comprise, a poloxamer with a polyoxypropylene molecular mass of approximately 900 g/mol and approximately 10% polyoxyethylene content, such as, without limitation, that sold under the trade name Pluronic® L31; a poloxamer with a polyoxypropylene molecular mass of approximately 900 g/mol and approximately 50% polyoxyethylene content, such as, without limitation, that sold under the trade name Pluronic® L35; a poloxamer with a polyoxypropylene molecular mass of approximately 1800 g/mol and approximately 50% polyoxyethylene content, such as, without limitation, that sold under the trade name Pluronic® P65; a poloxamer with a polyoxypropylene molecular mass of approximately 2400 g/mol and approximately 80% polyoxyethylene content, such as, without limitation, that sold under the trade name Pluronic® F88; sorbitan monolaurate such as, without limitation, that sold under the trade name Span® 20; sorbitan monopalmitate such as, without limitation, that sold under the trade name Span® 40; sorbitan monostearate such as, without limitation, that sold under the trade name Span® 60; sorbitan tristearate such as, without limitation, that sold under the trade name Span® 65; sorbitan monooleate such as, without limitation, that sold under the trade name Span® 80; polyoxyethylene sorbitan monolaurate, such as, without limitation, that sold under the trade name Tween® 20; polyoxyethylene sorbitan monopalmitate such as, without limitation, that sold under the trade name Tween® 40; polyoxyethylene sorbitan monostearate such as, without limitation, that sold under the trade name Tween® 60; polyoxyethylene sorbitan monooleate such as, without limitation, that sold under the trade name Tween® 80. A surfactant may comprise 4-(1,1,3,3-tetramethylbutyl)phenyl-polyethylene glycol, t-octylphenoxypolyethoxyethanol, polyethylene glycol tert-octylphenyl ether such as, without limitation, that sold under the trade name Triton® X-100.

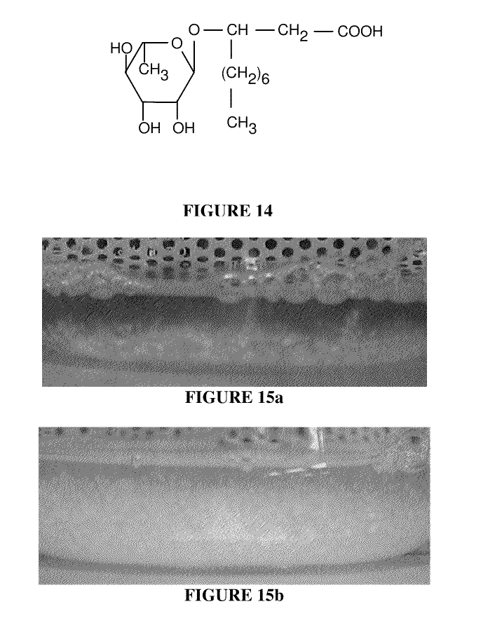

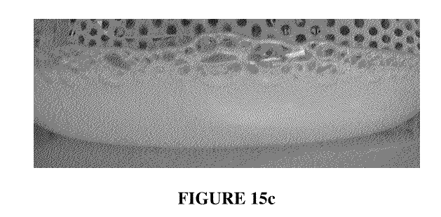

Rhamnolipids are glycolipids. Rhamnolipids comprise mono-rhamnolipids and di-rhamnolipids. FIG. 14 shows a diagram of a molecule of one non-limiting embodiment of di-rhamnolipid. FIG. 15 shows a diagram of a molecule of one non-limiting embodiment of mono-rhamnolipid. Rhamnolipids may comprise a combination of one or two rhamnose sugars and fatty acids. Rhamnolipids may comprise one or two molecules of rhamnose linked to a β-hydroxyalcanoic acid or a chain of two β-hydroxyalcanoic acids joined by an ester bond. Rhamnolipids may be produced by organisms including, without limitation, Pseudomonas aeruginosa, P. clororaphis, and Burkholderia pseudomallei. Pseudomonas aeruginosa strains that may be used to produce rhamnolipids include, without limitation, DSM 2874, ATCC 9027, ATCC 10145, and UG2.

In some concentrations, introduction of a surfactant may result in a change in the morphology of some of the T. reesei from filaments to generally spherical or ovoid pellets. In some concentrations, introduction of a surfactant may result in a change in the morphology of substantially all of the T. reesei from filaments to generally spherical pellets or generally ovoid pellets (“pellet form”). In some concentrations, introduction of a rhamnolipid may change the morphology of some portion of T. reesei from filaments to generally spherical pellets or generally ovoid pellets (“pellet form”). In some embodiments, introduction of a rhamnolipid may change the morphology of some portion of T. reesei resulting in bulges on filaments or at the tips of filaments.

The effects of several surfactants on the morphology of T. reesei were studied. The results are tabulated below in TABLE 1. TABLE 1 summarizes the observations of the effects of several surfactants regarding to flocculation or formation of pellets in T. reesei. Without limitation, in T. reesei, flocculation is correlated with pellet formation; that is, results in TABLE 1 showing tendencies toward cell flocculation indicate changes in the morphology of the T. reesei to pellet form. All surfactants listed in TABLE 1 were compared with 0.5 g/L and 1 g/L surfactant concentrations.

TABLE 1

| | Effective in | |
|---|---|---|
| | changing | |
| Surfactant | morphology? | Description |
| Pluronic ® F88 | YES | Increasing surfactant concentration |
| | | shows increased tendencies |
| | | towards cell flocculation |
| Pluronic ® P65 | YES | Increasing surfactant concentration |
| | | shows tendencies toward |
| | | increased flocculation |
| TWEEN ® 20 | YES | Increasing surfactant concentration |
| | | shows tendencies toward increased |
| | | flocculation |
| Rhamnolipids | YES | Surfactant concentrations greater than |
| | | 0.3 g/L yield nearly complete cell |
| | | pellet formation |
| Triton X-100 | YES | Formation of generally |
| | | uniform ~1 mm pellets |

Without limitation, surfactants that are effective in modifying the morphology of T. reesei comprise a rhamnolipid; a poloxamer with a polyoxypropylene molecular mass of approximately 2400 g/mol and approximately 80% polyoxyethylene content, such as, without limitation, that sold under the trade name Pluronic® F88; a poloxamer with a polyoxypropylene molecular mass of approximately 1800 g/mol and approximately 50% polyoxyethylene content, such as, without limitation, that sold under the trade name Pluronic® P65; and polyoxyethylene sorbitan monolaurate, such as, without limitation, that sold under the trade name Tween® 20. Another surfactant effective in modifying the morphology of T. reesei is 4-(1,1,3,3-tetramethylbutyl)phenyl-polyethylene glycol, t-octylphenoxypolyethoxyethanol, polyethylene glycol tert-octylphenyl ether such as, without limitation, that sold under the trade name Triton® X-100.

The effect of rhamnolipids on the morphology of T. reesei was studied. Experiments were conducted to study the effects of different concentrations of rhamnolipids on the morphology of T. reesei and on pellet formation in growing mixtures comprising T. reesei. Examples of experiments are described below. FIG. 5 shows vials corresponding to those in the experiments described in EXAMPLES 1-4 below. These examples are illustrative of certain embodiments. They are not to be construed as limiting unless otherwise stated.

TABLE 2

| Medium | g/l |
|---|---|
| Lactose | 10 |
| (NH4)₂SO₄ | 1.54 |
| KH₂PO₄ | 2 |
| MgSO₄•7H₂O | 0.3 |
| CaCl₂•2H₂O | 0.4 |
| NH₂CONH₂ | 0.3 |
| Trace Elements | mg/l |
| FeSO₄•7H₂O | 5 |
| MnSO₄•H₂O | 1.6 |
| ZnSO₄•7H₂O | 1.4 |
| CoCl₂ | 2 |

### Example 1

The effect of growing a fungus in a growth medium containing no rhamnolipids was tested. 100 ml of a growth medium was formed comprising the materials as listed in TABLE 2 and water sufficient to make up 100 ml of the medium. 100 ml of the formed growth medium was incorporated into a 250 ml shaker flask. Pre-cultured T. reesei fungal seeds were added to the growth medium in the 250 ml shaker flask to form a growing mixture. After a lag phase the fungus started to grow. The fungus was allowed to grow for several days. The flask was shaken with a shaker at 250 rpm.

FIG. 5 shows four vials. The vial 510 comprises the growing mixture resulting from the experiment described in EXAMPLE 1. The growing mixture in vial 510 was substantially uniform. Substantially all of the fungus in vial 510 was suspended in the growing mixture. The viscosity of the growing mixture in vial 510 was substantially higher than that of the initial growth medium.

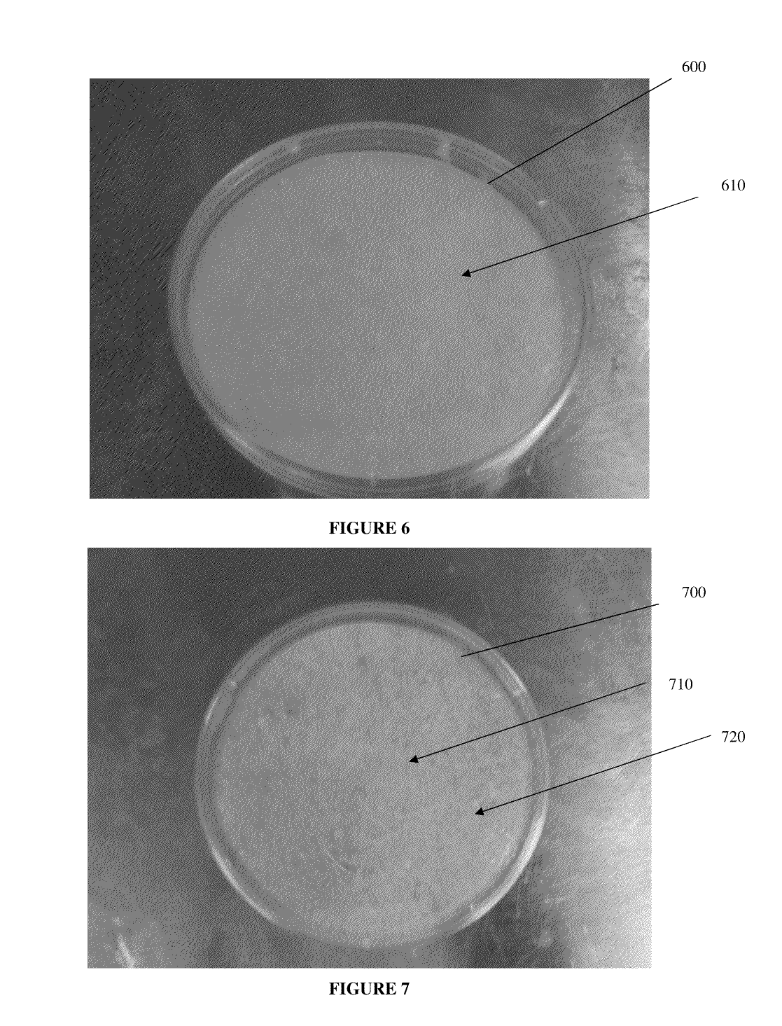

FIG. 6 shows a Petri dish 600 comprising the growing mixture resulting from the experiment described in EXAMPLE 1. The growing mixture 610 in Petri dish 600 is substantially uniform; no pellets are visible.

### Example 2

The effect of growing a fungus in a growth medium containing a 0.1 g/l concentration of rhamnolipids was tested. 100 ml of a growth medium was formed comprising the materials as listed in TABLE 2, 0.1 g/l of rhamnolipids, and water sufficient to make up 100 ml of the medium. 100 ml of the formed growth medium was incorporated into a 250 ml shaker flask. Pre-cultured T. reesei fungal seeds were added to the growth medium in the 250 ml shaker flask to form a growing mixture. The flask was shaken with a shaker at 250 rpm. After a lag phase the fungus grew. The fungus was allowed to grow for several days. Pellets appeared between the second and third day.

FIG. 7 shows a Petri dish 700 comprising the growing mixture resulting from the experiment described in EXAMPLE 2. The growing mixture 710 in Petri dish 700 is not uniform; it shows a few pellets 720.

FIG. 5 shows four vials. The vial 520 comprises a fraction of the growing mixture resulting from the experiment described in EXAMPLE 2. The growing mixture in vial 520 is not substantially uniform. The growing mixture in vial 520 shows a minor separation into a region 522 and region 524. Region 522 is substantially more transparent than region 524; this is evidence that region 522 contains a lower concentration of the fungus than region 524. Most of the fungus in vial 520 was suspended in the growing mixture with only a minor region 522 showing separation.

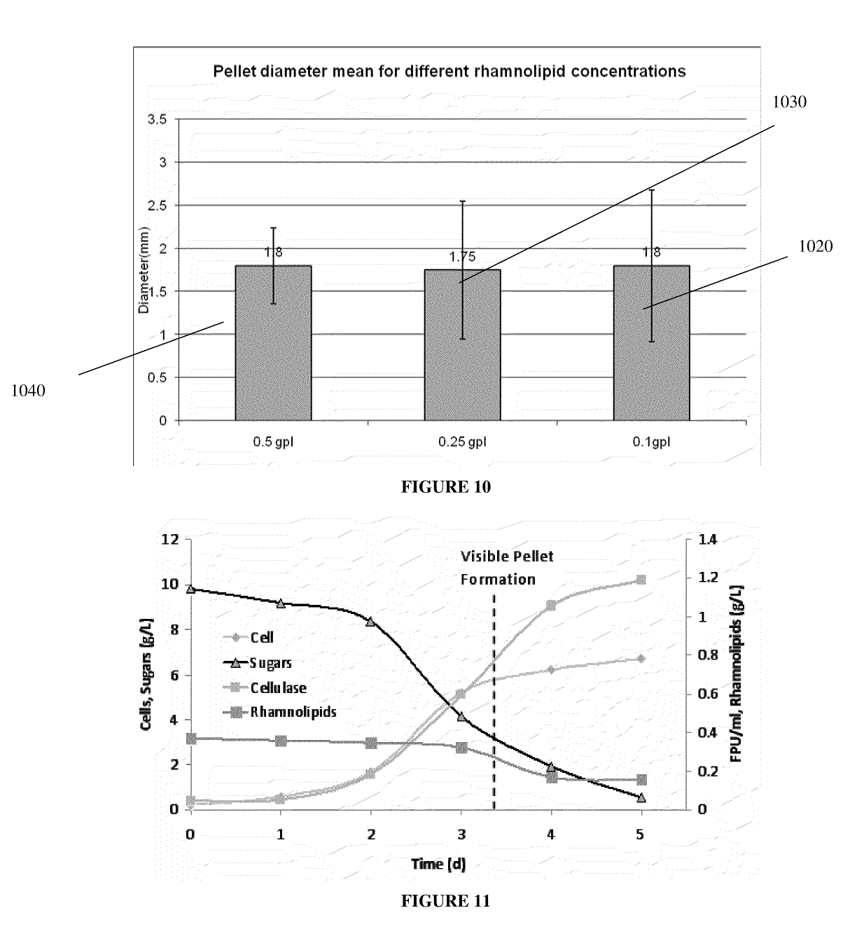

The column 1020 of FIG. 10 is a graph showing mean pellet diameter in millimeters for the pellets in the growing mixture resulting from the experiment described in EXAMPLE 2. As shown by column 1020, the mean pellet diameter was 1.8 mm. Also shown in column 1020, are a set of whisker lines indicating standard deviation of the pellet size.

### Example 3

The effects of growing a fungus in a growth medium containing a 0.25 g/l concentration of rhamnolipids were tested. 100 ml of a growth medium was formed comprising the materials as listed in TABLE 2, 0.25 g/l of rhamnolipids, and water sufficient to make up 100 ml of the growth medium. 100 ml of the formed growth medium was incorporated into a 250 ml shaker flask. Pre-cultured T. reesei fungal seeds were added to the growth medium in the 250 ml shaker flask to form a growing mixture. The flask was shaken with a shaker at 250 rpm. After a lag phase the fungus grew. The fungus was allowed to grow for several days. Pellets appeared between the second and third day.

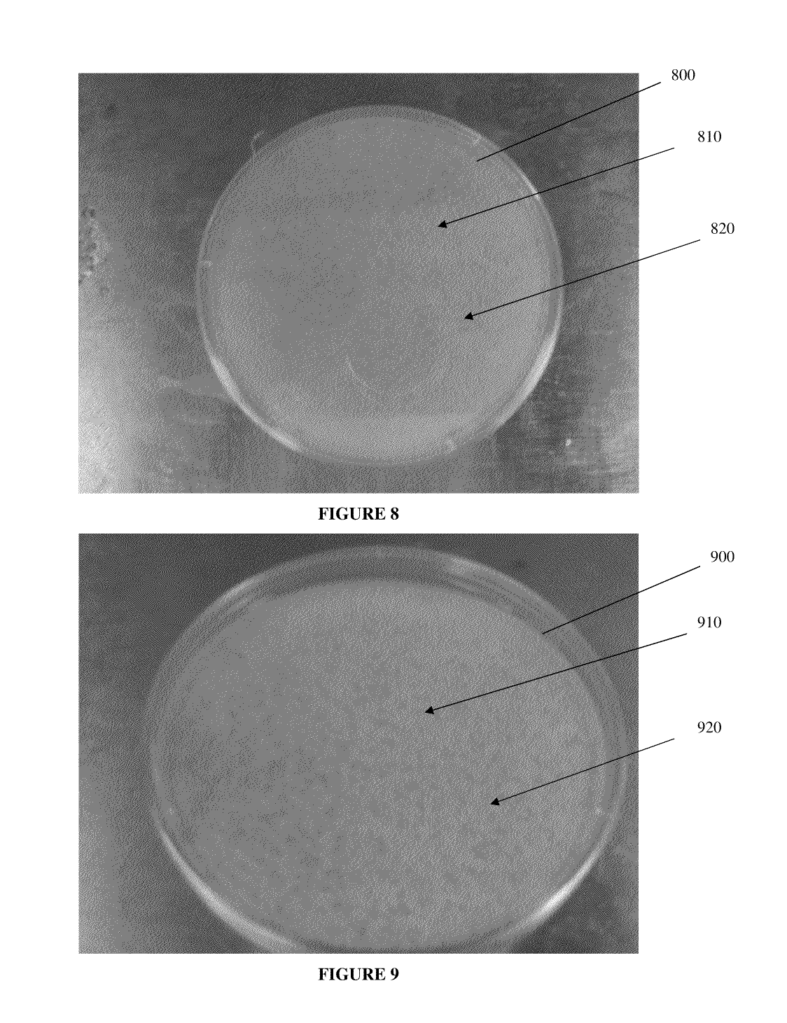

FIG. 8 shows a Petri dish 800 comprising the growing mixture resulting from the experiment described in EXAMPLE 3. The growing mixture 810 in Petri dish 800 is not uniform; it shows pellets 820 and few free cells.

FIG. 5 shows four vials. The vial 530 comprises the growing mixture resulting from the experiment described in EXAMPLE 3. The growing mixture in vial 530 is not substantially uniform. The growing mixture in vial 530 shows a minor separation into a region 532 and region 534. Region 532 is substantially more transparent than region 534; this is evidence that region 532 contains a lower concentration of the fungus than region 534. Most of the fungus in vial 530 was suspended in the growing mixture.

The column 1030 of FIG. 10 is a graph showing mean pellet diameter in millimeters for the pellets in the growing mixture resulting from the experiment described in EXAMPLE 3. As shown by column 1030, the mean pellet diameter was 1.75 mm. Also shown in column 1030, are a set of whisker lines indicating standard deviation of the pellet size. By comparison with column 1020, the whisker lines indicating standard deviation of the pellet size in column 1030, are smaller than the whisker lines indicating standard deviation of the pellet size in column 1020. This indicates that pellet size in EXAMPLE 3 was more uniform than was the pellet size in EXAMPLE 2.

### Example 4

The effects of growing a fungus in a growth medium containing a 0.5 g/l concentration of rhamnolipids were tested. 100 ml of a growth medium was formed comprising the materials as listed in TABLE 2, 0.5 g/l of rhamnolipids, and water sufficient to make up 100 ml of the growth medium. 100 ml of the formed growth medium was incorporated into a 250 ml shaker flask. Pre-cultured T. reesei fungal seeds were added to the growth medium in the 250 ml shaker flask to form a growing mixture. The flask was shaken with a shaker at 250 rpm. After a lag phase the fungus grew. The fungus was allowed to grow for several days. Pellets appeared between the second and third day.

FIG. 9 shows a Petri dish 900 comprising the growing mixture resulting from the experiment described in EXAMPLE 4. The growing mixture 910 in Petri dish 900 is not uniform; it shows many pellets 920.

FIG. 5 shows four 250 ml vials. The vial 540 comprises the growing mixture resulting from the experiment described in EXAMPLE 4. The growing mixture in vial 530 is substantially heterogeneous. The growing mixture in vial 540 shows a distinct separation into a region 542 and region 544. Region 542 is substantially transparent, while region 544 is not; this is evidence that region 542 contains a lower concentration of the fungus than region 544. All or almost all of the fungus in vial 540 precipitated from the growing mixture. The viscosity of the growing mixture in vial 540 was not substantially higher than that of the initial growth medium. Separation of the growing mixture into the growth medium and the fungus was readily possible using simple settling.

The column 1040 of FIG. 10 is a graph showing mean pellet diameter in millimeters for the pellets in the growing mixture resulting from the experiment described in EXAMPLE 4. As shown by column 1040, the mean pellet diameter was 1.8 mm. Also shown in column 1040, are a set of whisker lines indicating standard deviation of the pellet size. By comparison with column 1030, the whisker lines indicating standard deviation of the pellet size in column 1040, are smaller than the whisker lines indicating standard deviation of the pellet size in column 1030. This indicates that pellet size in EXAMPLE 4 was more uniform than was the pellet size in EXAMPLE 3. By comparison with column 1020, the whisker lines indicating standard deviation of the pellet size in column 1040, are smaller than the whisker lines indicating standard deviation of the pellet size in column 1020. This indicates that pellet size in EXAMPLE 4 was more uniform than was the pellet size in EXAMPLE 2. Taken together these data indicate that the pellet size uniformity increased with the concentration of the surfactant in EXAMPLES 2, 3, and 4.

### Example 5

The effect of growing a fungus in a growth medium containing a 0.5 g/l concentration of rhamnolipids was tested. One liter of a growth medium was formed comprising the materials as listed in TABLE 2, 0.5 g/l of rhamnolipids, and water sufficient to make up a liter of the growth medium. One liter of the formed growth medium was incorporated into a 2 liter fermentor. Pre-cultured T. reesei fungal seeds were added to the growth medium in the fermentor to form a growing mixture. The fermentor was stirred with an agitator at 150 rpm. After a lag phase the fungus grew.

The fungus was allowed to grow for several days as shown in FIG. 11. FIG. 11 shows the cellulase, sugar and rhamnolipid profile along with the cell growth. Pellets appeared between the third and fourth day. The resulting mixture contained primarily pellets and very few or no free cells.

The results of the experiments described in EXAMPLES 1-5 show a trend wherein the uniformity in the size of the pellets in the pellet form fungus was found to increase as surfactant concentration increased and the number of cells being modified into pellet form was found to increase as surfactant concentration increased. With higher surfactant concentrations, the fungus grown showed a greater fraction of cells developing into pellets and more uniform size distribution of the pellets formed. In some cases, pellet formation corresponded to lipid globules formed at the tips of filaments. FIG. 4 shows the presence of bulges 420 at the tip of the filaments 410; the bulges 420 are not present in EXAMPLE 1 in which the fungus was grown without rhamnolipids.

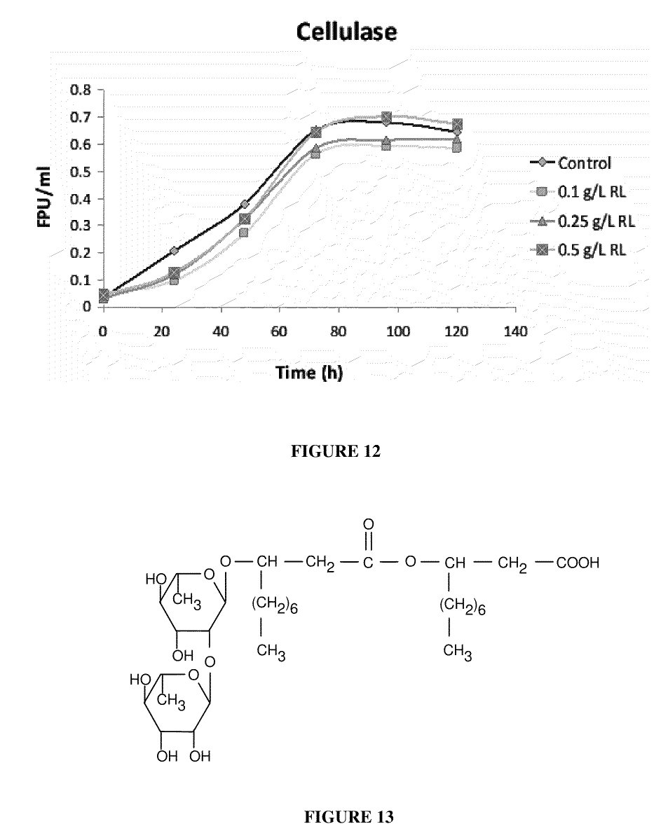

FIG. 12 is a graph of cellulase concentration as a function of time in each of the growing mixtures resulting from the experiments described in EXAMPLES 1-4.

In certain embodiments, such as, without limitation, those described in EXAMPLES 4 and 5, a growing mixture comprising a growth medium and a fungus primarily comprised of fungi having a pellet form morphology will not substantially increase in viscosity as the fungus grows. That is, the viscosity of the growing mixture just prior to harvest will be substantially similar to that of the growing mixture just after growth begins. In certain embodiments, such as, without limitation, those described in EXAMPLES 4 and 5, the viscosity of the growing mixture stays low throughout the period of fungal product production such that, the rate of oxygenation of the growing mixture by bubbling oxygen therethrough remains high enough to continue to promote fungal growth, and separation of the growing mixture into growth medium and fungus can be conducted quickly without complex separation methods.

### Example 6

The effect of growing a fungus in a growth medium containing Triton X-100 was tested. A culture was grown on a growth medium containing (g/L): (NH₄)₂SO₄ 1.4, KH₂PO₄ 2, MgSO₄.7H₂O 0.3, CaCl₂.2H₂O 0.4, urea 0.3, peptone 1.0, Tween-80 0.2, FeSO₄.7H₂O 0.005, MnSO₄.H₂O 0.0016, ZnSO₄.7H₂O 0.0014, CoCl₂ 0.002, and lactose 10.0. Initially the unadjusted pH was 5.5 after autoclaving.

Test cultures were grown in 50 mL volume in 250 mL flasks at 28° C. in a Que Orbital shaker (Model 4703, Parkersburg, W.Va., USA) at 250 rpm without pH control, initially at 5.5, for 7 days. Triton X-100 was tested at the following concentrations (g/L): 0.1, 0.3, 0.6, 0.8, 1.0, 1.5.

Cultures were followed for 7 days and visually checked for bulk morphology. Periodic monitoring was done aseptically by collecting a 2 mL representative sample every 24 hrs to measure cell concentration, enzyme activity, sugar concentration, and residual surfactant amounts.

As shown in FIG. 15b, without limitation, Triton was successful in producing a “pellet” growth when tested at concentrations greater than 0.3 g/L.

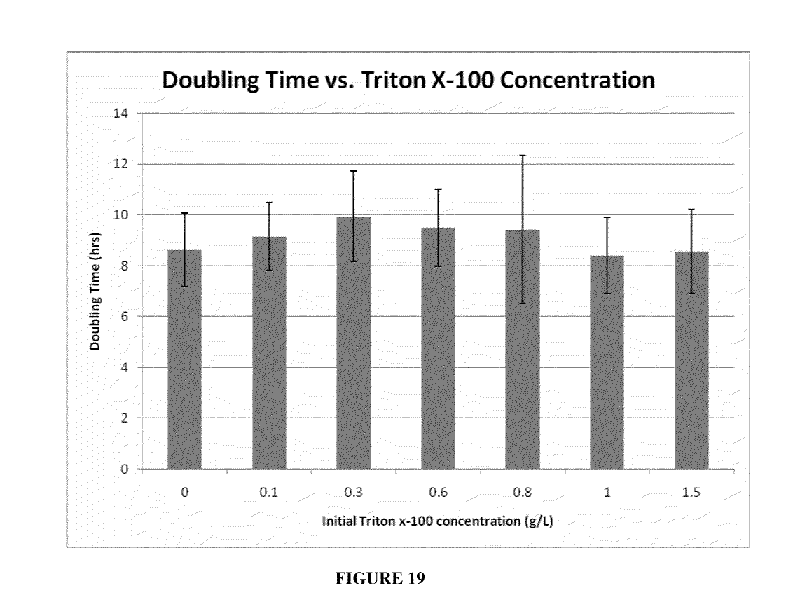

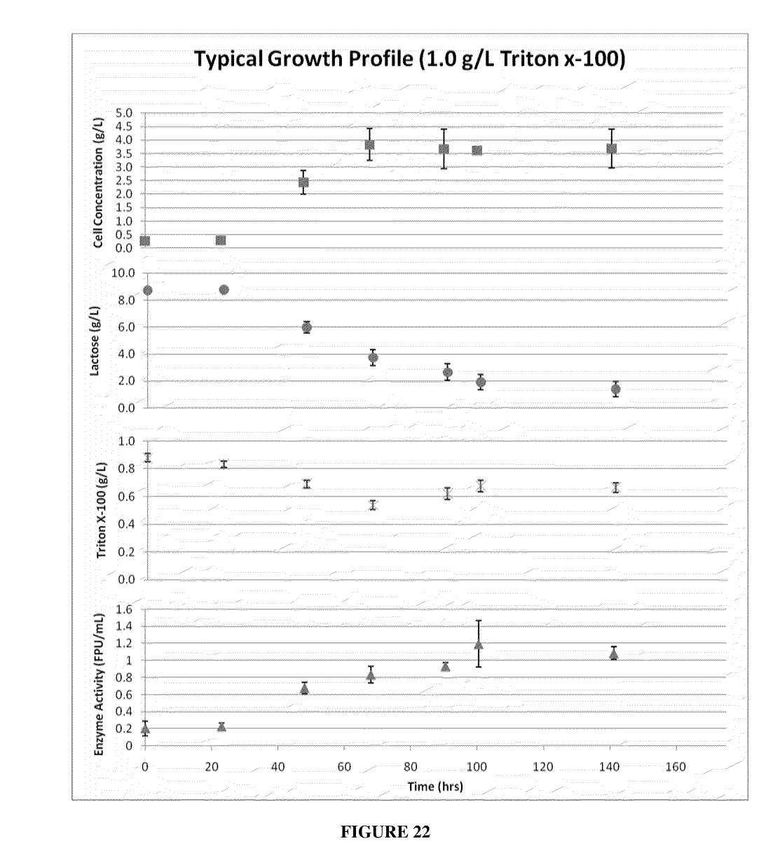

When compared to the surfactant free system, Triton produced a constant one-day lag for concentrations above 0.1 g/L. Following the lag phase, Triton influenced growth maintains a consistent doubling rate irrespective of the additive concentration. FIG. 19 displays the measured doubling times for varying concentrations of Triton. A single factor ANOVA indicates this data set has a p-value of 0.74 and the differences in growth rates are not significant enough to indicate a difference between the tested additive concentrations. The global average doubling time is 9.1±1.8 hrs. A typical growth profile indicating sugar consumption, cell concentration, enzyme production and Triton concentration is indicated in FIG. 22.

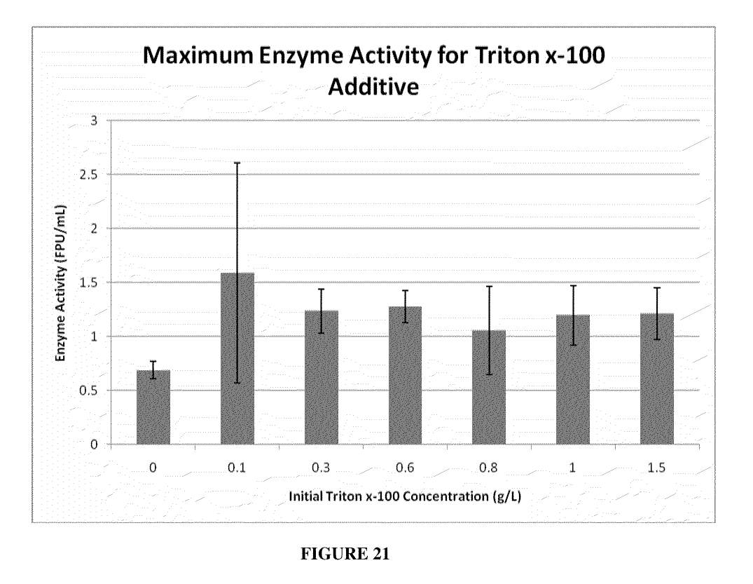

Enzyme production by T. reesei grown in the presence of Triton X-100 provided increased enzyme activity for additive concentrations in excess of 0.1 g/L. The effect of the additive does not appear to be a function of concentration, as shown in FIG. 21. For additive concentration in excess of 0.1 g/L, there is an average 73±12% increase in the enzyme activity.

### Example 7

The effect of growing a fungus in a growth medium containing rhamnolipids was tested. A culture was grown on a growth medium containing (g/L): (NH₄)₂SO₄ 1.4, KH₂PO₄ 2, MgSO₄.7H₂O 0.3, CaCl₂.2H₂O 0.4, urea 0.3, peptone 1.0, Tween-80 0.2, FeSO₄.7H₂O 0.005, MnSO₄.H₂O 0.0016, ZnSO₄.7H₂O 0.0014, CoCl₂ 0.002, and lactose 10.0. Initially the unadjusted pH was 5.5 after autoclaving.

Test cultures were grown in 50 mL volume in 250 mL flasks at 28° C. in a Que Orbital shaker (Model 4703, Parkersburg, W.Va., USA) at 250 rpm without pH control, initially at 5.5, for 7 days. Rhamnolipids were tested at the following concentrations (g/L): 0.1, 0.3, 0.6, 0.8, 1.0, 1.5.

Cultures were followed for 7 days and visually checked for bulk morphology. Periodic monitoring was done aseptically by collecting a 2 mL representative sample every 24 hrs to measure cell concentration, enzyme activity, sugar concentration, and residual surfactant amounts.

As shown in FIG. 15a, without limitation, rhamnolipids were successful in producing a “pellet” growth when tested at concentrations greater than 0.3 g/L.

Rhamnolipid additive was demonstrated to significantly modify the morphology and produce a stable pellet. To further investigate this attribute, shake flasks cultures of Trichoderma were grown at varying concentrations of rhamnolipids. A growth profile showing reducing sugar, cell concentration, enzyme activity and surfactant concentration is indicated in. As shown in, rhamnolipid additives yielded a correlation between the initial concentration of surfactant and the lag phase in exponential growth. When pairing the beginning of the exponential growth phase, contained within a 12 hr sampling window, with the concentration of rhamnolipids there is a 0.98 linear-correlation factor. However, the concentration of rhamnolipid additive may not be constant during the fermentation run and may decrease during cell growth.

Based on the cell concentrations, indicates the doubling time as a function of rhamnolipid additive. When the initial concentration of rhamnolipids is 0.3 g/L or less, there is no significant difference in mean doubling times, with an ANOVA p-value of 0.52. For additive concentrations of 0.6 to 1.0 g/L, there is no significant difference in doubling times, with an ANOVA p-value of 0.36. However, ANOVA results on the entire data set indicate a p-value of 0.04, for an a of 0.05 this result may indicate significant differences in the mean values. Based on the doubling times, there is a differentiation in cell growth rates occurring between the 0.3 and 0.6 g/L concentration.

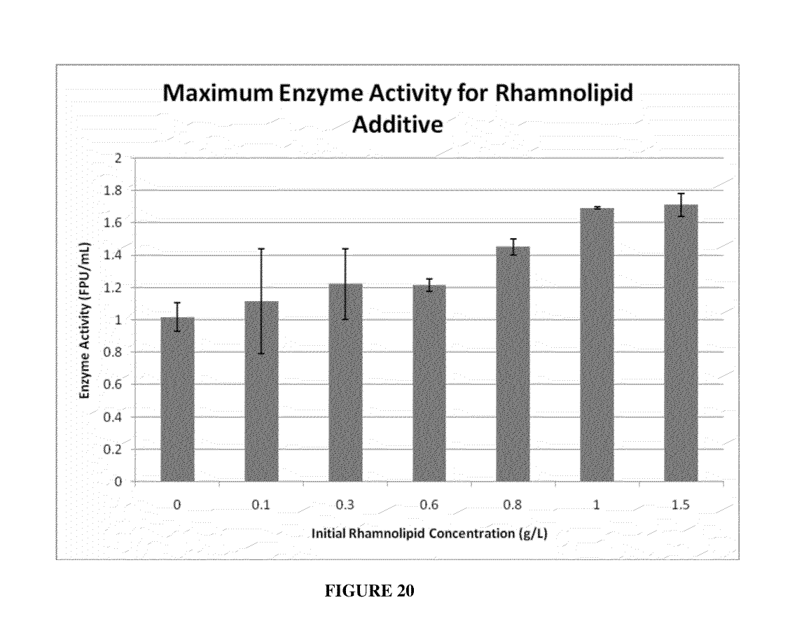

To provide an accurate comparison across multiple concentrations of rhamnolipid additive, a single medium was distributed among each of the shake flasks containing differing amounts of additive. With a fixed medium composition, each of the systems contains identical amounts of nitrogen, carbon and trace elements. The balanced medium should provide comparable enzyme yields across each system. Therefore, enzyme analysis on the rhamnolipid-influenced growth indicates an increasing maximum enzyme activity with additive concentration. The addition of rhamnolipids increased the enzyme activity up to 68±7.8%, in the case of the 1.5 g/L system. FIG. 20 indicates the maximum-recorded enzyme activity for each rhamnolipid concentration.

In certain embodiments, incorporating a surfactant, such as, without limitation, a rhamnolipid or Triton X-100, into a growing mixture comprising T. reesei may modify the morphology of the T. reesei and produce a growing mixture wherein the viscosity of the growing mixture just prior to harvest will be substantially similar to that of the growing mixture just after growth begins. In certain embodiments, the viscosity of the growing mixture just prior to harvest is low enough that separation of the growing mixture into growth medium and fungus is spontaneous.

In certain embodiments, harvesting of T. reesei that has been modified to a pellet form morphology may be very simple. In some embodiments, such as, without limitation, that of vial 540, T. reesei that has been modified to a pellet form morphology can be made to precipitate from the growing mixture. In some embodiments, such as, without limitation, that of vial 540, T. reesei that has been modified to a pellet form morphology will precipitate from the growing mixture spontaneously. In some embodiments, the fungus will precipitate from the growing mixture if the growing mixture may be allowed to rest for brief time. Where the fungus precipitates from the growing medium, the resulting mixture will be substantially separated into fungus and growth medium; that is, the mixture will have a very low medium to fungus ratio in lower regions and a very high medium to fungus ratio in upper regions. The very high medium to fungus ratio mixture is primarily growth medium and can be pulled off for harvesting purposes. In such embodiments, a very large fraction of the produced growth medium soluble cellulase may be extracted with the used growth medium while leaving a very large fraction of the produced fungus behind in the growth region.

FIG. 2 shows a method 200. The method 200 comprises harvesting growth medium and retaining the grown fungus for reuse. Method 200 comprises allowing the modified morphology fungus to precipitate from the growing mixture 210. In certain embodiment of 210, most of the modified morphology fungus is allowed to precipitate from the growing mixture. In certain embodiment of 210, a small fraction of the modified morphology fungus is allowed to precipitate from the growing mixture. The amount of fungus allowed to precipitate from the growing mixture in 210 is not critical. In some embodiments of 210, the modified morphology fungus is mostly a pellet form fungus. Method 200 comprises separating the growing mixture into the growth medium and the modified morphology fungus 220. In certain embodiments of 220, separation is performed by screening, filtering, vacuum filtering, centrifuging, or settling. Method 200 comprises retaining some amount of the modified morphology fungus 230. In certain embodiments of 230, most of the modified morphology fungus is retained. In certain embodiments of 230, retained fungus is left in the original growing region. In certain embodiments of 230, the retained fungus is maintained under the controlled growth conditions during harvesting. Controlled growth conditions refer to those conditions and protocols needed to protect the subject organisms from contamination. Maintenance of the integrity of controlled growth conditions makes certain anti-contamination procedures superfluous and may permit the reutilization of existing fungal samples without further anti-contamination procedures without introducing a substantial risk of contamination. Method 200 comprises introducing new growth medium to the retained fungus 240. In certain embodiments, introducing new growth medium to the retained fungus 240 creates a new growing mixture capable of producing desired fungal products without the need to re-grow substantial amounts of the fungus. In certain embodiments, the production of one or more subsequent batches of fungal products, such as, without limitation, cellulase, without having to re-grow substantial amounts of the fungus may result in additional efficiencies.

While the method of modifying fungal morphology has been described above in connection with the certain embodiments, it is to be understood that other embodiments may be used or modifications and additions may be made to the described embodiments for performing the same function of the method of modifying fungal morphology without deviating therefrom. Further, the method of modifying fungal morphology may include embodiments disclosed but not described in exacting detail. Further, all embodiments disclosed are not necessarily in the alternative, as various embodiments may be combined to provide the desired characteristics. Variations can be made by one having ordinary skill in the art without departing from the spirit and scope of the method of modifying fungal morphology. Therefore, the method of modifying fungal morphology should not be limited to any single embodiment, but rather construed in breadth and scope in accordance with the recitation of the attached claims.

What is claimed is:

1. A method of modifying fungal morphology comprising:
introducing an amount of a surfactant to a fungus, or to a growing mixture comprising a fungus, wherein said amount of a surfactant is sufficient to induce a change in the morphology of said fungus.

2. The method of claim 1, further comprising growing said fungus in a growth medium.

3. The method of claim 2, wherein said fungus has a naturally filamentous morphology.

4. The method of claim 3, wherein said surfactant comprises a member of the group consisting of
a rhamnolipid,
a poloxamer with a polyoxypropylene molecular mass of approximately 2400 g/mol and approximately 80% polyoxyethylene content,
a poloxamer with a polyoxypropylene molecular mass of approximately 1800 g/mol and approximately 50% polyoxyethylene content,
polyoxyethylene sorbitan monolaurate,
4-(1,1,3,3-tetramethylbutyl)phenyl-polyethylene glycol, t-octylphenoxypolyethoxyethanol, polyethylene glycol tert-octylphenyl ether, and combinations thereof.

5. The method of claim 4, wherein said fungus comprises, any strain of Trichoderma reesei, Trichoderma viride, Trichoderma koningii, Acremonium cellulolyticus, Aspergillus acculeatus, Aspergillus fumigatus, Aspergillus niger, Irpex lacteus, Penicillium funmiculosum, Phanerochaete chrysosporium, Schizophyllum commune, Sclerotium rolfsii, Sporotrichum cellulophilum, Talaromyces emersonii, Thielavia terrestris, Streptomyces sp., Thermoactinomyces sp., or Thermomonospora curvata.

6. The method of claim 5, wherein said change in the morphology of said fungus is a change to a substantially pellet form morphology.

7. The method of claim 6, wherein said surfactant comprises
a rhamnolipid, or
4-(1,1,3,3-tetramethylbutyl)phenyl-polyethylene glycol, t-octylphenoxypolyethoxyethanol, polyethylene glycol tert-octylphenyl ether.

8. The method of claim 7, wherein said fungus comprises any strain of Trichoderma reesei.

9. The method of claim 8, wherein the concentration of the surfactant in the growth mixture is equal to or greater than 0.1 gram per liter.

10. A method of producing cellulase comprising:
providing a growth medium, fungal cells, and a surfactant; and
mixing the fungal cells, the growth medium, and an amount of the surfactant to form a mixture, wherein said amount of the surfactant is an amount sufficient to induce a change in the morphology of the fungal cells.

11. The method of claim 10, wherein said fungal cells are of a fungus having a naturally filamentous morphology

12. The method of claim 11, wherein said surfactant comprises a member of the group consisting of
a rhamnolipid,
a poloxamer with a polyoxypropylene molecular mass of approximately 2400 g/mol and approximately 80% polyoxyethylene content,
a poloxamer with a polyoxypropylene molecular mass of approximately 1800 g/mol and approximately 50% polyoxyethylene content,
polyoxyethylene sorbitan monolaurate,
4-(1,1,3,3-tetramethylbutyl)phenyl-polyethylene glycol, t-octylphenoxypolyethoxyethanol, polyethylene glycol tert-octylphenyl ether, and combinations thereof.

13. The method of claim 12, wherein said fungus comprises, any strain of Trichoderma reesei, Trichoderma viride, Trichoderma koningii, Acremonium cellulolyticus, Aspergillus acculeatus, Aspergillus fumigatus, Aspergillus niger, Irpex lacteus, Penicillium funmiculosum, Phanerochaete chrysosporium, Schizophyllum commune, Sclerotium rolfsii, Sporotrichum cellulophilum, Talaromyces emersonii, Thielavia terrestris, Streptomyces sp., Thermoactinomyces sp., or Thermomonospora curvata.

14. The method of claim 13, wherein said change in the morphology of said fungal cells is a change to a substantially pellet form morphology.

15. The method of claim 14, wherein said surfactant comprises
a rhamnolipid, or
4-(1,1,3,3-tetramethylbutyl)phenyl-polyethylene glycol, t-octylphenoxypolyethoxyethanol, polyethylene glycol tert-octylphenyl ether.

16. The method of claim 15, wherein said fungus comprises any strain of Trichoderma reesei.

17. A growing mixture comprising:
a fungus;
a growth medium; and
an amount of a surfactant sufficient to induce a change in the morphology of said fungus.

18. The growing mixture of claim 17,
wherein said surfactant comprises a member of the group consisting of
a rhamnolipid,
a poloxamer with a polyoxypropylene molecular mass of approximately 2400 g/mol and approximately 80% polyoxyethylene content,
a poloxamer with a polyoxypropylene molecular mass of approximately 1800 g/mol and approximately 50% polyoxyethylene content,
polyoxyethylene sorbitan monolaurate,
4-(1,1,3,3-tetramethylbutyl)phenyl-polyethylene glycol, t-octylphenoxypolyethoxyethanol, polyethylene glycol tert-octylphenyl ether, and
combinations thereof;
wherein said fungus has a naturally filamentous morphology; and
wherein said change in the morphology of said fungus is a change to a substantially pellet form morphology.

19. The growing mixture of claim 18,
wherein said surfactant comprises a rhamnolipid, or
4-(1,1,3,3-tetramethylbutyl)phenyl-polyethylene glycol, t-octylphenoxypolyethoxyethanol, polyethylene glycol tert-octylphenyl ether; and
wherein said fungus comprises, any strain of Trichoderma reesei.

20. The growing mixture of claim 19, wherein said growing mixture comprises a concentration of a surfactant equal to or greater than 0.1 gram per liter.
